(19) United States
(12) Patent Application Publication
(10) Pub. No.: US 2012/0071794 A1
(43) Pub. Date: Mar. 22, 2012

(54) Robotic System for Delivering Energy for Treatment of Skin of a Subject

(75) Inventor: Ziv KARNI, Kfar Shmaryahu (IL)

(73) Assignee: Alma Lasers Ltd., Ceasarea (IL)

(21) Appl. No.: 12/885,658

(22) Filed: Sep. 20, 2010

(51) Int. Cl. A61B 18/20; A61B 18/18; A61N 7/00; A61B 19/00

(52) U.S. Cl. 601/2; 606/130; 606/9; 606/33

(57) ABSTRACT

A robotic system for delivering energy for medical or cosmetic treatment of a region of skin of a subject includes a multi-axis robotic arm mounted on a base and terminating at a holder. An applicator for delivering energy to the skin is mounted in the holder. The holder has at least three range sensors generating outputs of a distance to at least three spaced apart locations on the surface of the skin. A controller associated with the robotic arm and the range sensors processes the outputs from the at least three range sensors to determine an orientation of the holder relative to the skin, and actuates the robotic arm to adjust an orientation of the holder such that an orientation of the applicator relative to the skin is maintained within a predefined range of desired angles.

### FIELD AND BACKGROUND OF THE INVENTION

The present invention relates to skin treatment systems and, in particular, it concerns a robotic system for delivering energy for medical or cosmetic treatment of a region of skin of a subject.

U.S. Pat. No. 7,083,611 to Lemchen discloses a highly schematic robotic system including a plurality of dermatological devices for treatment of facial skin. Lemchen provides no guidance as to how the robotic system should be implemented.

US patent application publication no. 2008/0033410 to Rastegar et al. discloses a robotic system for performing laser wound debridement, employing a multi-axis robotic arm operated by a control system which employs 3D vision processing from a pair of cameras. The laser treatment head operates in a non-contact mode, spaced by at least 10 centimeters from the skin, performing a scanning irradiation pattern from each laser head stopping location.

US patent application publication no. 2005/0193451 to Quistgaard et al. discloses an articulated arm for providing load-balanced or robotic functionality for medical procedures, particularly ultrasound therapy. Where used in contact with the body, Quistgaard proposes combining a location tracking system associated with the treatment head together with motion sensors mounted on the body to derive relative position of the treatment head and the body.

In principle, the use of a robotic tool offers potential advantages for treatment of the skin, potentially relieving the operator of part of the workload of sequentially treating a large number of closely spaced treatment locations uniformly distributed over a desired treatment region. In practice, the aforementioned systems present significant problems for implementation, particularly for types of treatment in which an applicator must contact the skin surface and be maintained at a required angle relative to the local skin surface. Specifically, even a relatively small error in mapping of the body surface or tracking of body movements could cause unacceptable force to be exerted on the body by the robot system and/or could result in loss of contact or misalignment of the applicator, thereby greatly reducing effectiveness of the treatment. Even if available, systems for such accurate mapping of the body surface and tracking of motion would be complex and expensive, rendering the system of limited commercial applicability. Automated implementations of such devices encounter a number of problems inherent to working with the skin, including that the surface is irregular (due to contours), inconsistent (due for example to underlying muscle vs. fat vs. bone) and dynamically unpredictably changing (for example, due to breathing or movement of the subject).

There is therefore a need for a robotic system for delivering energy for medical or cosmetic treatment of a region of skin of a subject which would provide a simple and low cost solution to ensure correct positioning of an applicator relative to the skin surface.

### SUMMARY OF THE INVENTION

The present invention is a robotic system for delivering energy for medical or cosmetic treatment of a region of skin of a subject.

According to an embodiment of the present invention there is provided, a robotic system for delivering energy for medical or cosmetic treatment of a region of skin of a subject, the system comprising: (a) a multi-axis robotic arm mounted on a base and terminating at a holder; (b) an applicator for delivering energy to the skin, the applicator being mechanically associated with the holder; (c) at least three range sensors associated with the holder and deployed to generate outputs indicative of a distance to a corresponding at least three spaced apart locations on the surface of the skin; and (d) a controller associated with the robotic arm and the range sensors, the controller including at least one processor, the controller being configured to: (i) process the outputs from the at least three range sensors to determine an orientation of the holder relative to the skin, and (ii) actuate the robotic arm to adjust an orientation of the holder such that an orientation of the applicator relative to the skin is maintained within a predefined range of desired angles.

There is also provided according to an embodiment of the present invention, a method for delivering energy for medical or cosmetic treatment of a region of skin of a subject, the system comprising: (a) providing a multi-axis robotic arm mounted on a base and terminating at a holder; (b) providing an applicator for delivering energy to the skin, the applicator being mechanically associated with the holder; (c) providing at least three range sensors associated with the holder and deployed to generate outputs indicative of a distance to a corresponding at least three spaced apart locations on the surface of the skin; (d) processing the outputs from the at least three range sensors to determine an orientation of the holder relative to the skin, and (e) actuating the robotic arm to adjust an orientation of the holder such that an orientation of the applicator relative to the skin is maintained within a predefined range of desired angles.

According to a further feature of an embodiment of the present invention, the controller is configured to actuate the robotic arm so as to move the holder along a substantially orbital path about a point of contact between the applicator and the skin.

According to a further feature of an embodiment of the present invention, the predefined range of desired angles lies within 20 degrees of perpendicular.

According to a further feature of an embodiment of the present invention, the range sensors are non-contact range sensors.

According to a further feature of an embodiment of the present invention, the applicator is retractably mounted relative to the holder so as to retract under contact pressure with the skin, and wherein one of the range sensors is implemented as a position sensor deployed for sensing a position of the applicator relative to the holder.

According to a further feature of an embodiment of the present invention, the applicator is retractably mounted relative to the holder, and wherein the head further comprises a resilient biasing arrangement deployed so as to bias the applicator against the skin.

According to a further feature of an embodiment of the present invention, the resilient biasing arrangement includes a counterweight deployed to substantially cancel variations in the bias due to the weight of the applicator.

According to a further feature of an embodiment of the present invention, the applicator is referred to as a first applicator, and further comprising a second applicator, wherein the first and second applicators are configured to be interchangeably mounted relative to the holder, and wherein the first and second applicators are each configured to provide a distinct treatment modality selected from the group consisting of: laser light; non-laser light; RF irradiation; microwave irradiation; ultrasound treatment; and mechanical manipulation.

According to a further feature of an embodiment of the present invention, the applicator is part of an RF irradiation treatment system.

According to a further feature of an embodiment of the present invention, the base of the robotic arm is mounted slidably on a substantially linear track.

According to a further feature of an embodiment of the present invention, there is also provided at least one user-operable control for controlling a position of the holder, and wherein the controller is configured to: (a) allow a user to maneuver the holder so as to bring the holder into alignment with a location on the skin; and (b) receive from the user a point designation input designating the location on the skin as a boundary point at least partially defining a treatment region.

According to a further feature of an embodiment of the present invention, alignment of the holder with the location on the skin is defined by alignment of a visible light beam projected from the holder with the point on the skin without requiring contact with the skin, and wherein the controller is further configured, after receipt of the point designation input, to actuate the robotic arm to move the applicator into contact with the skin at the boundary point.

There is also provided, according to an embodiment of the present invention, a method for designating a treatment region on the skin of a subject for therapeutic or aesthetic treatment, the method comprising the steps of: (a) allowing manual manipulation of a treatment head of a robotic skin treatment system so as to bring the treatment head into alignment with a location on the skin; and (b) receiving a point designation input designating the location on the skin as a boundary point at least partially defining a treatment region.

According to a further feature of an embodiment of the present invention, the alignment is defined by alignment of a visible light beam projected from the treatment head with the point on the skin without requiring contact with the skin, wherein the method further comprises, after receipt of the point designation input, moving the treatment head into contact with the skin at the boundary point.

According to a further feature of an embodiment of the present invention, the steps of allowing manual manipulation and receiving a point designation input are repeated so as to designate a plurality of boundary points, the method further comprising: (a) defining a boundary line derived from the plurality of boundary points; and (b) defining a scanning pattern bounded by the boundary line, thereby defining a treatment region on the skin.

### BRIEF DESCRIPTION OF THE DRAWINGS

The invention is herein described, by way of example only, with reference to the accompanying drawings, wherein:

FIG. 1 is a schematic isometric view of a robotic system, constructed and operative according to an embodiment of the present invention, for delivering energy for medical or cosmetic treatment of a region of skin of a subject;

FIG. 2 is an enlarged view of a robotic arm and applicator of the robotic system of FIG. 1;

FIG. 3 is a block diagram of the system of FIG. 1;

FIG. 4 is a more detailed block diagram of a holder (block 18) associated with the robotic arm of FIG. 3;

FIG. 5 is a detailed isometric view of an exemplary implementation of the holder of FIG. 4;

FIG. 6 is a bottom isometric view of the holder of FIG. 5;

FIG. 7 is a partially disassembled view of the holder of FIG. 5 revealing components of a bias adjustment mechanism according to an embodiment of the present invention;

FIG. 8 is a partially disassembled view of the holder of FIG. 5 revealing components of a counterweight arrangement according to an embodiment of the present invention;

FIG. 9 is a flow diagram illustrating an overall programming sequence for setting parameters of treatment according to an aspect of the present invention;

FIG. 10 is a flow diagram illustrating a procedure for designating boundaries of treatment regions according to an aspect of the present invention;

FIG. 11 is a more detailed flow diagram illustrating a procedure for designating a point on the boundary of a treatment region according to an aspect of the present invention;

FIGS. 12A and 12B are schematic side views illustrating the principles of applicator orientation self-correction functionality according to an aspect of the present invention;

FIGS. 13A-13F are a sequence of schematic side views illustrating stages during designation of a boundary of a treatment region according to an aspect of the present invention;

FIGS. 14A-14E are a sequence of schematic side views illustrating implementation of one pass of a treatment sequence according to an aspect of the present invention; and

FIGS. 15A-D are schematic illustrations of a number of motion templates according to aspects of the present invention.

### DESCRIPTION OF THE PREFERRED EMBODIMENTS

The present invention is a robotic system for delivering energy for medical or cosmetic treatment of a region of skin of a subject.

The principles and operation of robotic systems according to the present invention may be better understood with reference to the drawings and the accompanying description.

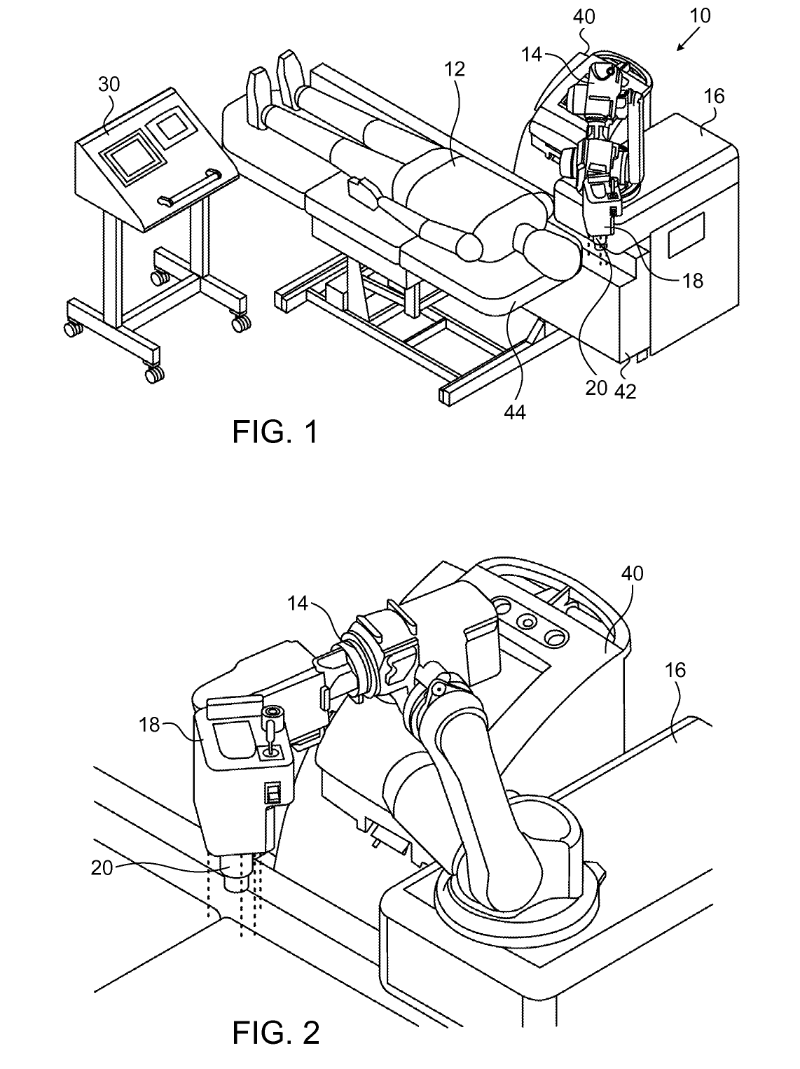

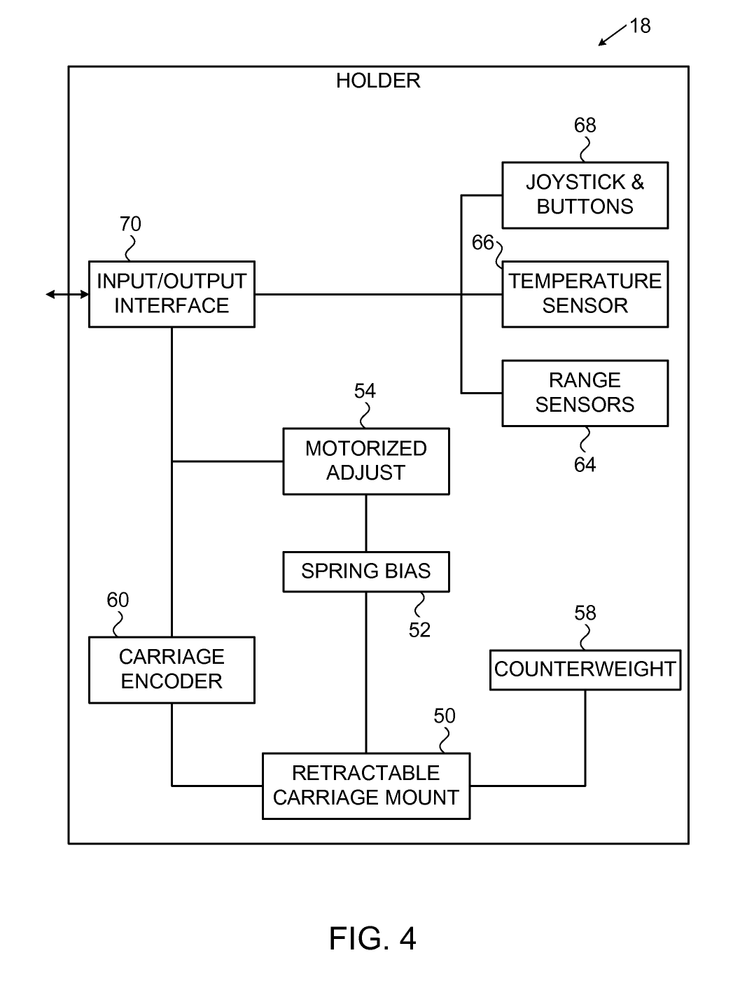

Referring now to the drawings, FIGS. 1-15 illustrate various aspects of the structure and operation of an embodiment of a robotic system, generally designated 10, for delivering energy for medical or cosmetic treatment of a region of skin of a subject 12. In general terms, robotic system 10 has a multi-axis robotic arm 14 mounted on a base 16 and terminating at a holder 18 which receives an applicator 20 for delivering energy to the skin. For convenience, holder 18 and applicator 20 may be referred to herein collectively as the “treatment head” of the robotic device. According to an aspect of the present invention, holder 18 has at least three range sensors (identified collectively by reference numeral 64 in FIG. 4 and individually by reference numerals 64a, 64b and 64c in FIG. 6) deployed to generate outputs indicative of a distance to corresponding spaced apart locations on the surface of the skin. A controller 24, associated with robotic arm 14 and range sensors 64, includes at least one processor 26 and typically other components such as data storage 28. Controller 24 is configured to process the outputs from range sensors 64 to determine an orientation of holder 18 relative to the skin, and to actuate robotic arm 14 to adjust an orientation of holder 18 such that an orientation of applicator 20 relative to the skin is maintained within a predefined range of desired angles.

At this point, it will be immediately apparent that the range-sensor-based self-correction of orientation of holder 18 relative to the skin surface according to this aspect of the present invention provides distinct advantages. Specifically, since adjustment is performed locally as a function of the measured orientation relative to the skin, the complex requirements of the prior art to achieve accurate modeling of the skin surface and tracking of even minor body movements are greatly relaxed. This feature, preferably in combination with various additional features to be described below, allows particularly simple and intuitive user operation and minimizes the need for complex and computationally heavy three-dimensional modeling and tracking capabilities. This and other advantages of the present invention will be better understood in view of the following description.

Before addressing the features of the present invention in more detail, it will be helpful to define certain terminology as used herein in the description and claims. Firstly, reference is made to “delivering energy for treatment of the skin”. “Energy” in this context is used to refer to any type of electromagnetic radiation or mechanical excitation which is at least partially absorbed within one or more layer of the skin and is effective to achieve or contribute towards a desired physiological effect. As particularly preferred but non-limiting examples, the present invention may be implemented to advantage with energy application systems for achieving fat removal, hair removal, skin resurfacing and/or skin tightening.

Reference is made to the “modality” of the energy treatment and corresponding energy delivery system. “Modality” in this context refers to the class of energy delivered to the skin, typically designated in terms of the type of electromagnetic radiation or mechanical energy used, and in some cases, may refer more specifically to a particular range of operating conditions (wavelength, energy density etc.) effective to achieve a distinct effect. Each modality may be applied alone, or a single applicator may provide two or more modalities, simultaneously or sequentially. Although not limited to these modalities, particularly preferred modalities to be used with the present invention include: RF radiation, including high frequency (HF or “shortwave”), medium wave (MW) and long wave (LW); microwave radiation; laser or non-laser light (infrared, visible or ultraviolet); ultrasound vibration; and mechanical manipulation. Examples of suitable energy treatment systems for these modalities are commercially available from ALMA LASERS Ltd. (Israel) under the tradenames: ACCENT® (RF radiation); SOPRANO® (laser light); PIXEL® CO2 (laser light), HARMONY® (non-laser light) and ACCENT® ULTRA (including ultrasound vibration plus mechanical manipulation). Patent documents disclosing applicators potentially relevant for use in embodiments of the present invention include, but are not limited to, U.S. Pat. No. 7,630,774, PCT patent application publication no. WO 2007/099545 and co-pending US patent application publication no. US 2009/0254068, PCT patent application publication no. WO 2007/099546 and co-pending US patent application publication no. US 2009/0012585, PCT patent application publication no. WO 2009/095894, and U.S. patent application Ser. No. 12/834,033, all of which are assigned to the assignee of the present invention. The present invention relates primarily to modalities and corresponding devices in which an applicator of the energy delivery system is placed in contact with, or in close proximity to, the skin surface.

In certain implementations of the present invention, the system is described as being “modular”. The term “modular” in this context is used to indicate the capability of interchanging between energy systems of different modalities and/or different combinations of modalities. Thus, according to an aspect of the present invention, robotic system 10 can be used interchangeably with energy systems and corresponding applicators for delivering two or more modalities selected from laser light, non-laser light, RF irradiation, ultrasound treatment and mechanical manipulation.

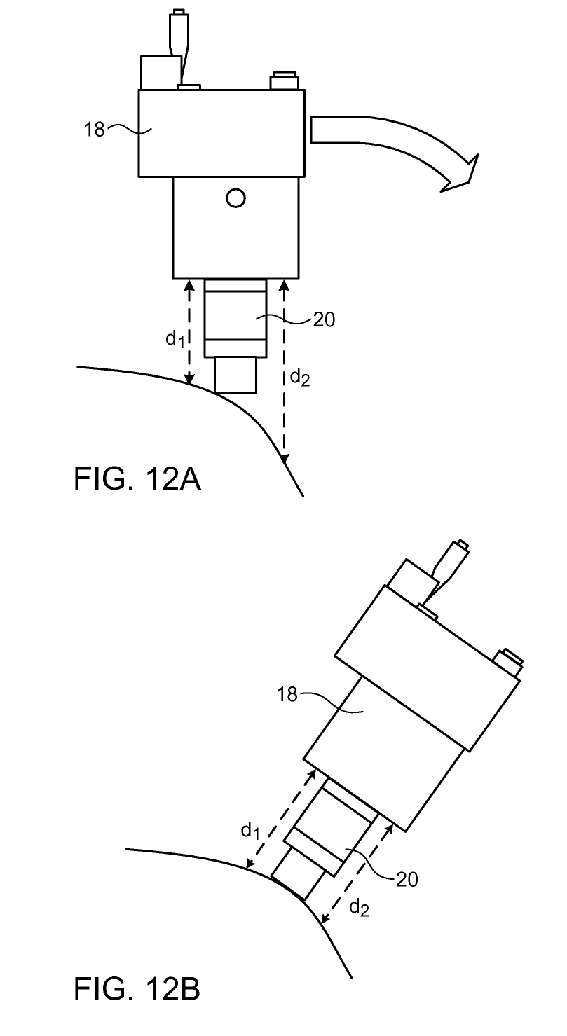

Reference is made below to motion along an “orbital path about a point”. For the purpose of this description and the appended claims, the term “orbital” is used to refer to motion which approximates to an arcuate path about the given point, and in which an inward-pointing alignment is maintained. Thus, if applicator 20 undergoes orbital motion about a point of contact with the skin, the end result will be that the applicator is rotated relative to its original position but substantially maintains the original point of contact from before the motion. This point may be better understood by reference to FIGS. 12A and 12B, and the accompanying description below.

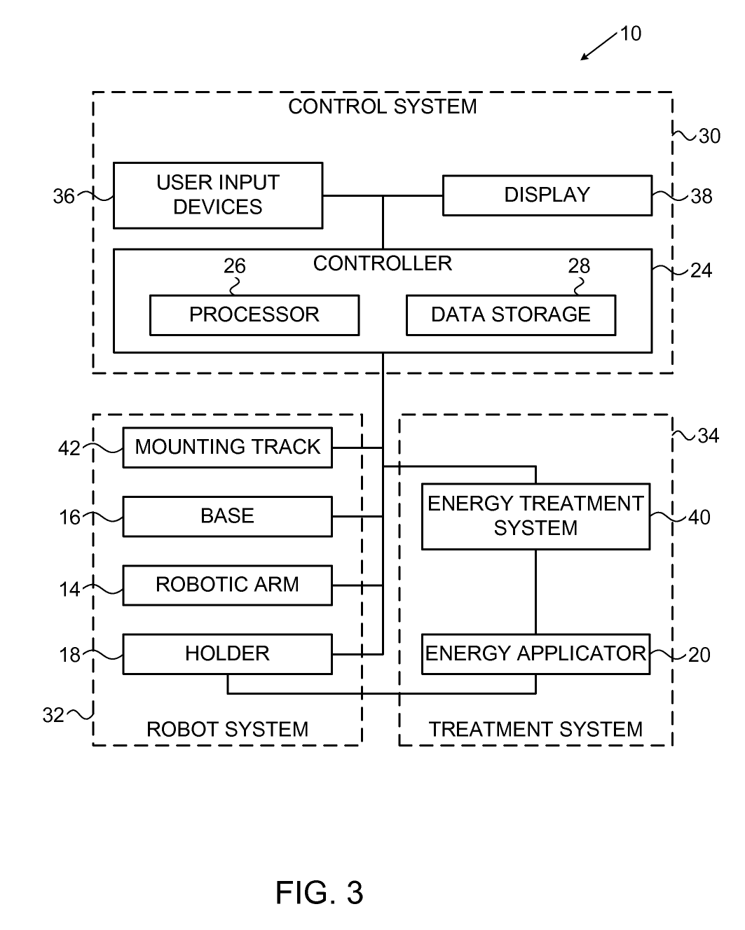

Turning now to the features of the present invention in more detail, FIG. 3 shows an overview of an embodiment of the present invention. For simplicity of presentation, the system is shown subdivided into three primary subsystems: a control system 30, a robot system 32 and a treatment system 34. It will be noted that this subdivision reflects what is believed to be a logical and convenient subdivision for implementation, but in no way limits the scope of the invention.

Control system 30, also shown in FIG. 1, includes controller 24, user input devices 36 (typically in the form of various keys and/or pointing devices) and one or more display 38. The display may optionally provide at least some of the user input functionality, for example, when implemented as a touch-screen.

Controller 24 may be implemented with one or more general purpose processors implemented with suitable interface modules to communicate with components of robot system 32 and treatment system 34, and operating under the control of software programmed to implement the various processes which are further detailed in the following disclosure. Alternatively, some or all of these processes may be performed by dedicated hardware or firmware, the implementations of which will be clear to one ordinarily skilled in the art on the basis of the following disclosure of the processes.

Treatment system 34 includes energy applicator 20 which operates together with a corresponding energy treatment system 40, also shown in FIG. 1. Energy treatment system 40 may be an otherwise conventional energy treatment system of the chosen modality, such as for example the ALMA LASERS products mentioned above. Applicator 20 may be a conventional applicator, but is more preferably an applicator with a modified form factor adapted to be received within holder 18, as will be described below. In other respects, applicator 20 is most preferably structurally and functionally closely analogous to conventional applicators designed for manual operation.

As mentioned earlier, certain implementations of the present invention are modular in that they allow interchangeable use of treatment systems 34 with different modalities. In this case, the overall combined system is highly unusual in that it preferably includes applicators for two or more modalities, or combinations of modalities, chosen from RF irradiation, laser light, non-laser light, ultrasound and mechanical manipulation in which the applicators of different modalities have similar form factors and are interchangeably received within the same holder 18.

Turning now to robot system 32, robotic arm 14 may be conveniently implemented as a standard 5-axis robotic arm of dimensions suitable for spanning the width of the human body and for approaching the far side of the body at a desired angle. In some cases, fewer than 5 axes may be sufficient, and certainly more than 5 axes may be used. In order to reduce the required dimensions of the robotic arm, the base 16 of the robotic arm is preferably mounted so as to be displaceable along a substantially linear track 42 deployed along one side of the treatment table 44 (see FIG. 1). As a result, base 16 can be repositioned for procedures performed in the upper or lower body, and coverage of the robotic arm spanning the width of the body is typically sufficient. According to an optional implementation (not shown), base 16 may be deployed so as to extend above the subject's body, thereby facilitating access to both sides of the body.

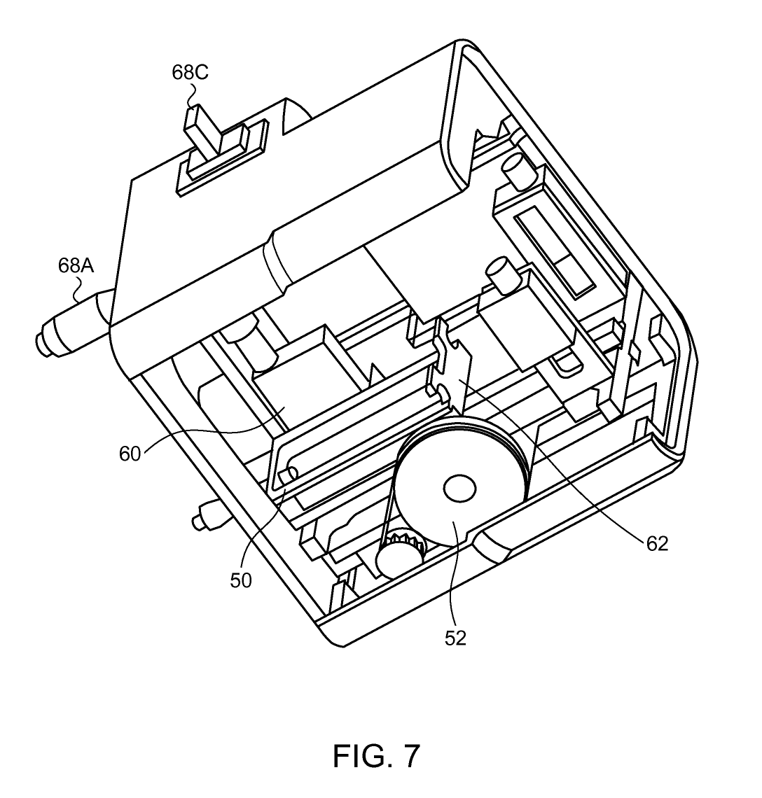

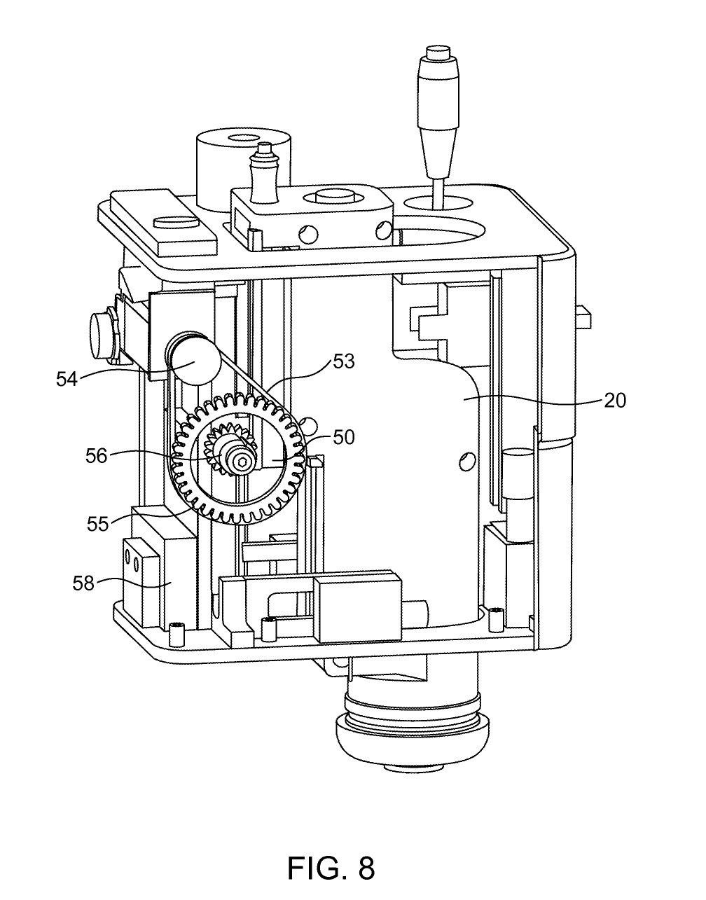

Various features of a preferred embodiment of holder 18 are illustrated in FIGS. 4-8. Referring first to FIG. 4, holder 18 includes a retractable carriage mount 50 to which applicators 20 are attached interchangeably. Carriage mount 50 is retractable relative to the casing of holder 18, and is biased to a forward (extended) position by a spring bias arrangement 52. Optionally, spring bias arrangement 52 may be adjustable by a motorized adjustment mechanism 54 which either increases or decreases the pre-tension in bias arrangement 52. In a particularly preferred exemplary implementation illustrated in FIG. 7, spring bias arrangement 52 is implemented as a coil spring deployed so as to provide a torque to an axle 56, visible in FIG. 8, which carries a gear wheel engaged with a rack (not shown) integrated with carriage mount 50. Motorized adjustment mechanism 54 is here implemented as a motor deployed to rotate an annular gear 55 via a toothed belt 53. Annular gear 55 forms the outer anchor of the coil spring (not shown). Thus, rotation of annular gear 55 increases or decreases the rotational tension in the coil spring, and hence the rotational bias applied to axle 56. The ability to adjust the spring bias, and hence the contact pressure of the applicator against the skin, is particularly valuable due to the modularity of the system, i.e., when switching between different modalities. Specifically, when performing light or RF irradiation, significant contact pressure is not required, and light contact pressure will typically be preferred. During application of ultrasound or mechanical manipulation, on the other hand, effective mechanical engagement of the applicator with the skin is essential to the proper operation of the applicator, requiring a more significant contact pressure.

Although the spring bias arrangement illustrated here is believed to be particularly advantageous due to its simplicity and reliability, it will be clear that many other implementations are possible, including but not limited to, arrangements of helical springs, leaf springs, air springs, electromagnetic springs or actuators.

A further feature illustrated in FIGS. 4 and 8 is a counterweight 58 deployed to substantially cancel variations in the spring bias due to the weight of applicator 20. In the preferred exemplary implementation best illustrated in FIG. 8, counterweight 58 is slidingly mounted so as to slide along a direction parallel to motion of that of carriage mount 50 and is engaged with the opposite side of the gear wheel (or another gear wheel) on axle 56 so as to ensure equal and opposite linear motion of the counterweight and the applicator. Clearly, many alternative implementations may be used. For example, it will be clear that an equivalent effect may be achieved using a larger counterweight moving through a smaller distance than the applicator. Optionally, motion of the counterweight may be independently controlled by a dedicated actuator. However, the simple mechanical linkage illustrated here is believed to be particularly advantageous due to its simplicity and reliability.

The effect of counterweight 58 is to roughly balance the weight of applicator 20 so that the contact pressure of the applicator against the skin is defined by spring bias arrangement 52 and not significantly affected by the orientation of holder 18. In some implementations, during switching of applicators from one modality to another, it may be necessary to replace or modify the counterweight in order to accurately match differing weights of the different applicators. This may be achieved by providing a quick-release weight adjustment block (not shown) which can be added or removed from the counterweight during switching of modalities. According to a more preferred option, the interchangeable applicators may be modified by addition of weights where necessary in order to bring each applicator up to within a predefined tolerance from a standardized weight, allowing a single counterweight to provide effective compensation for the weight of each applicator.

Holder 18 also includes a carriage encoder 60 for monitoring the position of carriage mount 50. In the preferred exemplary implementation shown in FIG. 7, carriage encoder 60 is implemented as a non-contact range sensor (e.g., laser rangefinder) which measures the distance from its fixed location within the holder housing to a flange 62 attached to carriage mount 50. Clearly, other types of encoder could also be used, such as an optical or other angular encoder mounted on axle 56 or other types of contact or non-contact linear encoders. In some cases, an arrangement of microswitches indicating whether carriage mount 50 is currently within or outside a target range of positions may also provide sufficient functionality.

It should be noted that the combination of a sliding retractable carriage mount 50, spring bias arrangement 52 and carriage encoder 60 facilitates accurate control of the contact pressure of applicator 20 while greatly relaxing the stringency of requirements for tracking of the skin contour by the robotic system. Specifically, if the robotic system was carrying a rigidly mounted applicator, an error of as little as a few millimeters could cause a sudden increase in contact pressure which might result in pain or injury to the patient. By using a spring biased retractable mount, a predefined range of clinically acceptable contact pressures is translated into a relatively long retraction motion, typically in excess of 30 millimeters, and more preferably at least 50 millimeters. Regulation of the contact pressure within this range is achieved by relatively coarse position adjustment according to the output of encoder 60. For example, if the subject inhales, causing his or her chest to move upwards, carriage mount 50 is momentarily displaced to a further retracted position against spring bias arrangement 52, and the retraction is sensed by encoder 60. The controller 24 then actuates robotic arm 14 so as to move holder 18 gradually further from the body surface, thereby returning the retraction mechanism to a preferred central part of the operating range. Conversely, if the skin moves away from holder 18, the spring bias arrangement 52 moves carriage mount 50 forwards to maintain contact between applicator 20 and the skin, and the extension is sensed by encoder 60. Controller 24 then actuates robotic arm 14 to move holder 18 closer to the body surface until carriage mount 50 again returns to a central part of its operating range. This arrangement is effective to maintain a substantially constant contact pressure. Even a considerable transient error in the positioning of holder 18 relative to the skin surface will only result in a small deviation from the intended contact pressure, and will quickly be corrected based on the encoder output. In the event of extreme retraction beyond some defined safety threshold, a fault is indicated and operation is typically interrupted.

Turning now to the remaining features of holder 18 as illustrated in FIG. 4, the holder preferably includes a set, typically at least three, of range sensors 64 deployed to measure a distance between the holder and corresponding spaced apart points on the surface of the skin. The range sensors are preferably non-contact range sensors, and most preferably, laser rangefinders. Such devices are low cost components readily available from commercial sources.

Parenthetically, it will be appreciated that, given known dimensions of energy applicator 20, for as long as the end of the applicator is in contact with the skin, the measurement of encoder 60 is also indicative of the distance of holder 18 from the skin at the point of contact of the applicator. As a result, variant embodiments may dispense with encoder 60, instead relying upon range sensors 64 that provide information about the holder position relative to the skin, and deriving the applicator position therefrom. Alternatively, in the reverse direction, the output of encoder 60 may optionally be used to derive a range measurement of the holder from the skin, thereby supplementing data from the other range sensors, or allowing one of the range sensors to be omitted.

A further component of preferred implementations of holder 18 is a non-contact temperature sensor 66. Non-contact temperature sensors are well known, and operate by sensing thermal infrared emissions from the skin surface, represented here schematically by a dashed line. This sensor provides important safety functions, monitoring the skin surface during treatment to ensure that heating of the skin is kept within an acceptable range for each type of treatment.

FIG. 6 illustrates a preferred exemplary layout of range sensors 64 and temperature sensor 66. In this case, three range sensors 64 are roughly equally angularly spaced around the functional treatment axis of applicator 20, thereby providing information as to the plane of the skin surface encountered by the applicator. Temperature sensor 66 is deployed with its sensing field of view adjacent to the treatment region.

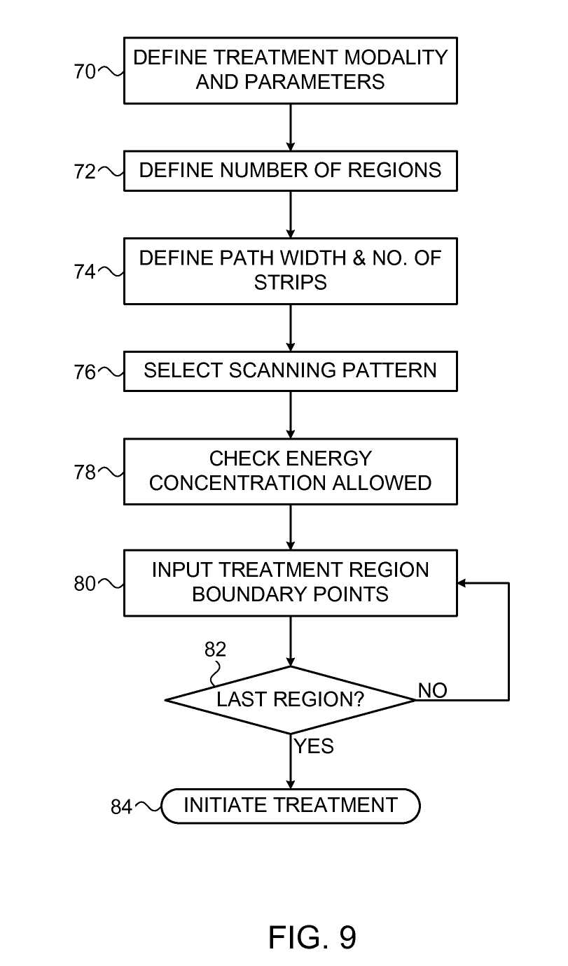

Additional features of holder 18 include one or more user input devices 68 such as a joystick and associated buttons, primarily for maneuvering the holder. Although operation of system 10 is primarily controlled from the control system user interface, it is a particularly preferred feature of an aspect of the present invention that programming of a region of skin to be treated is performed at least in part by directly maneuvering holder 18 to designate points defining at least one boundary of the treatment region. This procedure, discussed further below with reference to FIGS. 9-13F, is most easily performed when manipulating holder 18 by use of user controls on the holder itself.

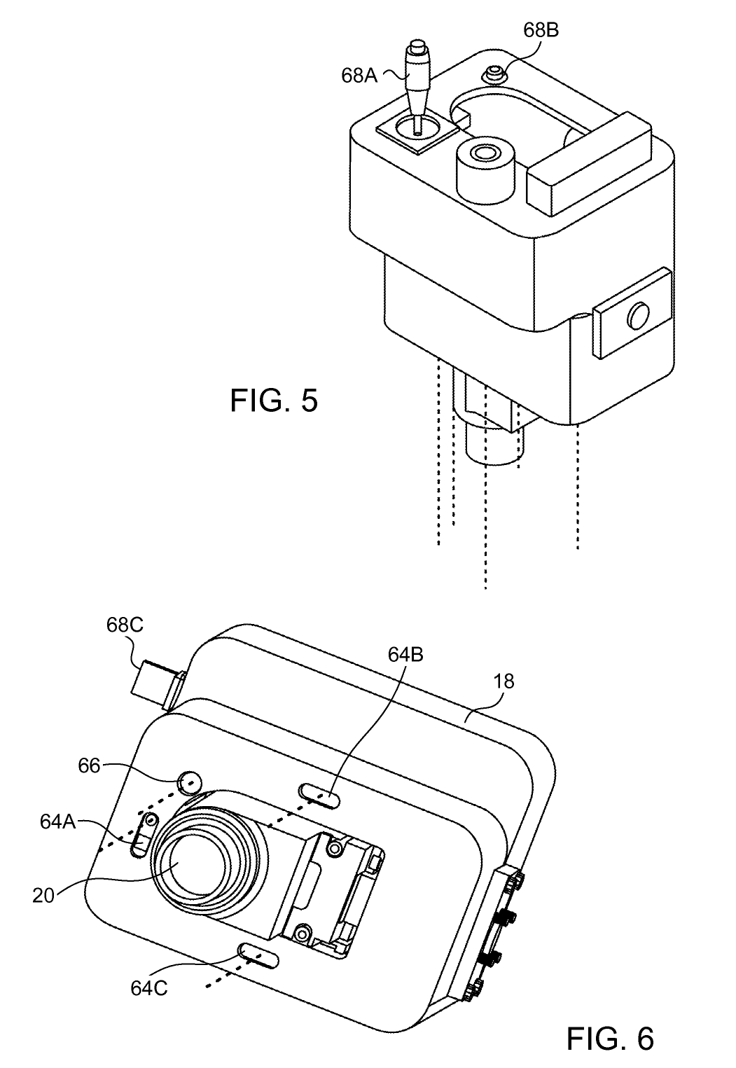

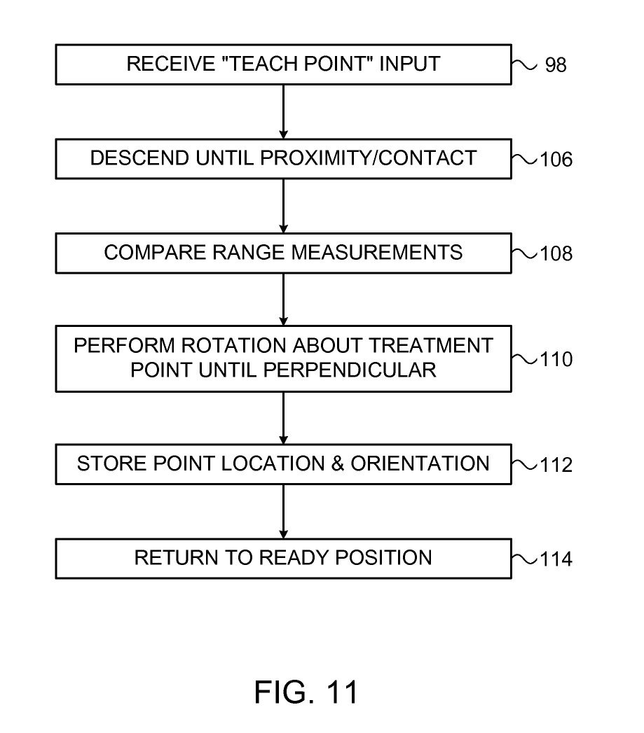

In the preferred exemplary implementation shown in FIGS. 5-7, the user input devices include a joystick 68a, a boundary point designation button 68b, and an up/down rocker switch 68c. In this case, joystick 68a is preferably used as the input for moving holder 18 to and fro in a horizontal plane above the patient, rocker switch 68c is used for raising and lowering holder 18 vertically, and button 68b is used to initiate a boundary point acquisition sequence which will be described below with reference to FIG. 11. According to a preferred feature of an embodiment of the invention, joystick 68a is a dual-function input device which, when an additional control button is depressed, operates to tilt 18 so as to incline at a desired angle to the vertical. This can be particularly useful for accurate designation of boundary points on inclined regions of the body surface, as will be described further below. Clearly, alternative user interfaces for manipulating holder 18 may be used in an analogous manner. For example, a handle provided with suitable transducers for sensing a manually applied force may be used to provide force-responsive motion, giving the user the impression that he or she is directly moving the holder. Many such user interfaces may be used, and are considered generally equivalent for the purposes of implementing the present invention.

The various inputs and sensors of holder 18 as well as the motorized bias adjustment operate in direct communication with control system 30 via an input/output interface 70, which is typically a wired interface with wires passing via robotic arm 14, but may alternatively be a wireless communication interface.

Turning now to FIG. 9, this provides an overview of operation of the system according to a preferred implementation of the invention. Firstly, at step 70, the system determines, by user inputs and/or automatically, the desired treatment modality and the required operating parameters, such as operating power. For automatic determination of data, each applicator may be provided with coded information (for example, by inclusion of a barcode or RFID with a corresponding reader in holder 18 or by distinctive mechanical features of each applicator which engage corresponding microswitches). Alternatively, direct communication between controller 24 and energy treatment system 40 may either allow the controller to directly recover details of a programmed treatment regime from the energy treatment system, or may allow setting of those parameters via the controller.

The user then defines the remaining treatment parameters in a sequence of steps, not necessarily in this order, exemplified as follows. Step 72 illustrated here allows the user to define the number of regions of skin to be treated in the session. Each region may be a distinct patch of skin in a different region of the body. Alternatively, where a single extended region of the skin with complex curvature is to be treated, it is advantageous to subdivide the region into a number of subregions over which the surface can be approximated by a simpler curvature.

Step 74 defines a path width and a number of strips. According to an aspect of the present invention, each treatment region is defined by mapping out one boundary line of the surface of the patient's skin and then defining a scanning pattern to be performed starting (or finishing) along the mapped boundary line. For example, if the applicator has an effective treatment coverage of 10 millimeters diameter for a given applicator position, a path width, and corresponding step between strips, of 10 millimeters will typically be used. If 5 strips (i.e., scanning passes) are used, the total treated area with have a width of 50 millimeters starting along the line of the mapped boundary and extending in the treatment direction therefrom. In some implementations, the boundary points and boundary line may define an approximate border between a currently treated skin region and an untreated (or not currently treated) region. In this case, the actual treatment scanning path is typically offset from the boundary line by half the effective treatment diameter of the applicator. Alternatively, the boundary line may define the actual line of motion of the applicator along one boundary of the treatment region. In this case, the border between treated and untreated skin will lie beyond the “boundary line” by roughly half the effective treatment coverage diameter, subject to the exact scanning pattern.

The boundary line may extend in any orientation and may be defined on any region of the body. In some cases, it may be advantageous to define the boundary line extending along a long axis of a limb, thereby encountering the minimum curvature during the longer motions of the scanning pattern.

Step 76 defines a scanning pattern for use in the treatment. Depending on the modality and treatment parameters, various different scanning patterns may be preferred. A number of examples of scanning paths, each designated 77, are illustrated schematically in FIGS. 15A-15D. The available patterns may include, but are not limited to, some or all of the following:
A simple reciprocating scanning motion (e.g., FIG. 15A) with single or multiple passes over each strip.
A zigzag or weaving motion (e.g., FIG. 15C) in which the scanning movement is further modified by a zigzag or undulating transverse wave motion.
A helical loop motion (e.g., FIG. 15D) in which the scanning movement is further modified by a circular or elliptical motion.
A line-mode in which the applicator moves to-and-fro along a given line of the scanning pattern for a predefined time period before moving on to the next line.
Stepped motion in which the applicator is actuated while briefly held stationary and is then moved to the next treatment location along a scan pattern such as that of FIG. 15A, optionally with some delay between successive actuations.

The spacing of the strips for the scanning pattern is typically dictated by the size of the treatment area under the applicator, such as is illustrated schematically in the enlarged view FIG. 15B, so that a desired degree of coverage of the area is achieved, according to the parameters of the required treatment. In certain cases, a particular desired effect can best be achieved by a treatment pattern which does not achieve full area coverage of the region being treated.

According to a preferred option, the parameters defined in the preceding steps are then processed at step 78 to verify that the overall energy concentration that will be delivered according to the defined operating parameters falls within acceptable limits from a safety point of view.

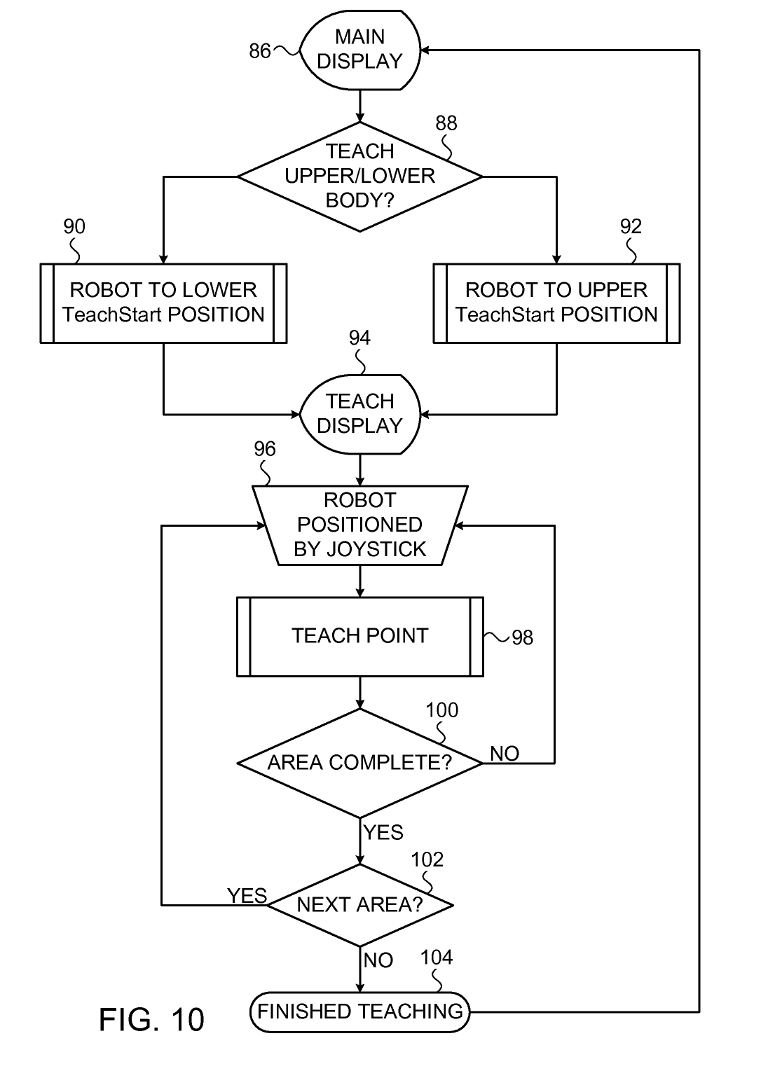

At step 80 a number of points are input by the user to define a boundary of a treatment region, and at step 82, this procedure is repeated until each treatment region has been defined. Optionally, in a contiguous-regions mode, the last point designated on the boundary of a first region may serve as the first point of the next region, thereby assuring proper alignment of two adjacent regions to achieve a larger area coverage. Other patterns of adjacent regions may also be implemented automatically or semi-automatically. Steps 80 and 82 are described in more detail with reference to FIGS. 10 and 11 below. Then, at step 84, the treatment may be initiated. The control system then actuates the robot system to move along the treatment paths defined by the aforementioned parameters, and synchronously actuates the energy treatment system to deliver energy while the applicator is in the appropriate locations or motion to effect the treatment.

FIG. 10 exemplifies a sequence employed to designate a boundary of each treatment region. At 86, the system provides a main display from which, at step 88, the user selects whether the region to be defined is in the upper or lower part of the body. According to the user input, the system then either moves the robot along mounting track 42 to lower start position (step 90) or upper start position (step 92) and provides the user with the “teach” boundary designation display (step 94).

The holder is then positioned by use of the joystick 68a through displacement and/or tilting until it is aligned with a first boundary point for the first treatment region (step 96) and the boundary point designation button 68b is depressed to “teach” the desired point (step 98). The system then executes the steps shown in FIG. 11 to record the position and orientation of the boundary point, as detailed below.

In order to facilitate correct alignment of holder 18 aligned with the desired boundary point, certain preferred implementations of the invention employ one range sensor 64a having a visible wavelength laser beam projected parallel to the central axis of the energy applicator 20 so as to designate the intended boundary point. The lateral offset between the visible laser beam and the actual central axis of the energy applicator is a known distance, and is corrected by a lateral movement of holder 18 during the boundary point designation process described below. The remaining range sensors 64b and 64c are implemented using invisible IR wavelengths so as to avoid confusion to the user. All of the light beams are represented schematically here by dashed lines. Alternatively, a dedicated laser pointer may be provided to facilitate correct alignment of the holder with the desired boundary point in a manner similar to that described above.

As mentioned earlier, some implementations of the invention allow manual adjustment of the inclination of holder 18 during the teaching process. This is particularly valuable for achieving reliable and accurate designation of a boundary point on a region of the body surface steeply inclined to the horizontal, such as the side of the torso.

After designation of each point, the system checks whether the given region boundary has yet been fully defined (step 100) and returns to step 96 when required. In most cases, it has been found effective to define three boundary points along a given line, although it is clearly possible to employ a larger number if desired. Where the region is small or relatively flat, two boundary points may also be sufficient.

After completion of the boundary designation for a region, step 102 checks whether additional regions need to be defined and, when necessary, again returns the user to step 96 with a suitable prompt to designate boundary points for the next region. After designation of sufficient boundary points for all regions to be treated, the designation process ends at step 104.

FIG. 11 illustrates the response of the system on receipt of a “teach point” input according to step 98 above, reproduced here as the first step. After lateral correction for the offset of the designation laser beam, the robotic system then moves holder 18 downwards (or along the axis of the applicator, if it differs from vertical) until the applicator 20 is brought into contact with, or reaches a desired proximity to, the desired boundary point (step 106). At this point, the system compares the measurements d₁ and d₂ from the range sensors 64 (step 108), as shown in FIG. 12A, thereby allowing the system to determine the local orientation of the skin surface at the given boundary point. Although shown here schematically in two dimensions for clarity, the calculations and corrections are performed in three dimensions, including a third measurement d₃ (not shown). The controller then actuates the robotic arm so as to move holder 18 along a path approximating to an orbital path about the given boundary point until the applicator lies within a predefined range of desired angles relative to the skin surface (step 110), as illustrated in FIG. 12B. The predefined range of angles typically corresponds roughly to the local perpendicular to the skin, for example ±20 degrees, and more preferably ±10 degrees, thereby rendering d₁, d₂ and d₃ all roughly equal. It will be noted however that treatments which can be delivered (or possibly which require specifically to be delivered) at angles differing further from the perpendicular also fall within the scope of the invention. Then, at step 112, the location and orientation of the designated boundary point are stored, for later use in determining the boundary line and corresponding path to be followed by the holder during treatment. Holder 18 is then returned to its ready position (step 114), typically raised up above the body of the patient, to facilitate maneuvering in a horizontal plane to the next boundary point.

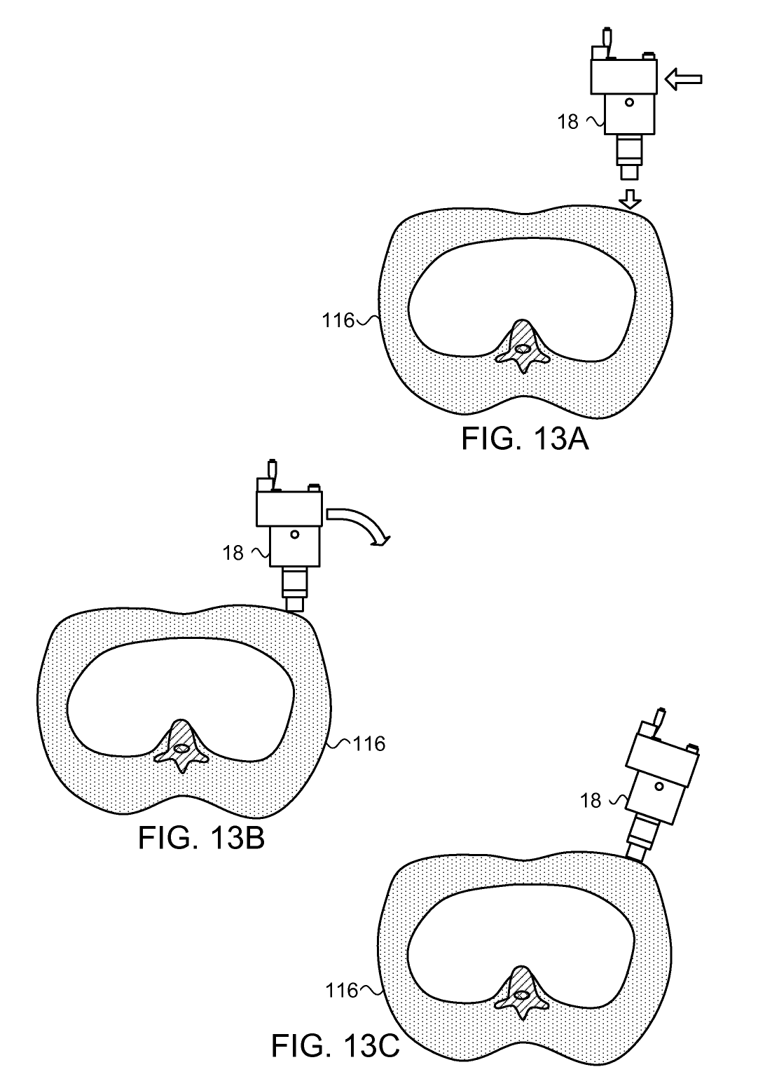

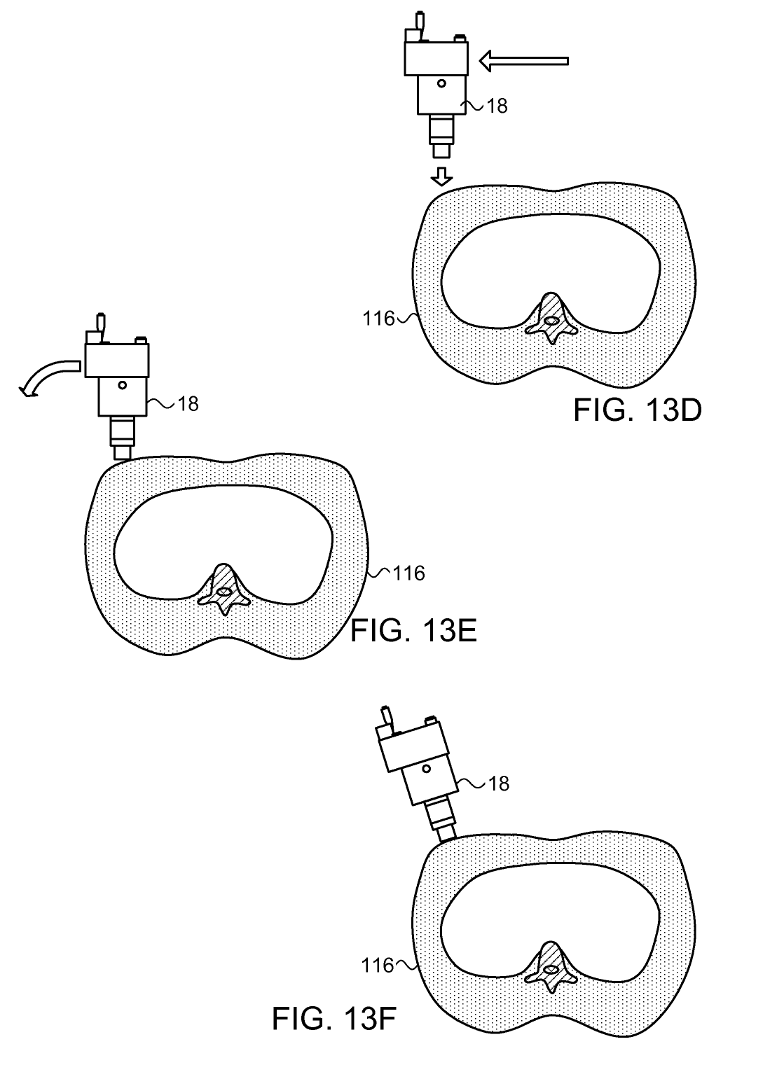

An example of designating a boundary line according to this aspect of the present invention is illustrated schematically in FIGS. 13A-13F. For simplicity of representation, the body of the subject as viewed in cross-section in a transverse plane has been approximated by a shaded shape 116. Specifically, FIGS. 13A-13C illustrate designation of a first boundary point in which holder 18 is maneuvered horizontally to be aligned with a first desired boundary point (FIG. 13A), descends to the position of FIG. 13B and then self-corrects its orientation while maintaining alignment with the desired boundary point, as shown in FIG. 13C. FIGS. 13D-13F illustrate a similar sequence for defining a boundary point at the opposite side of the body 116. This process is typically supplemented by designation of at least one additional point near the midpoint of the boundary (not shown).

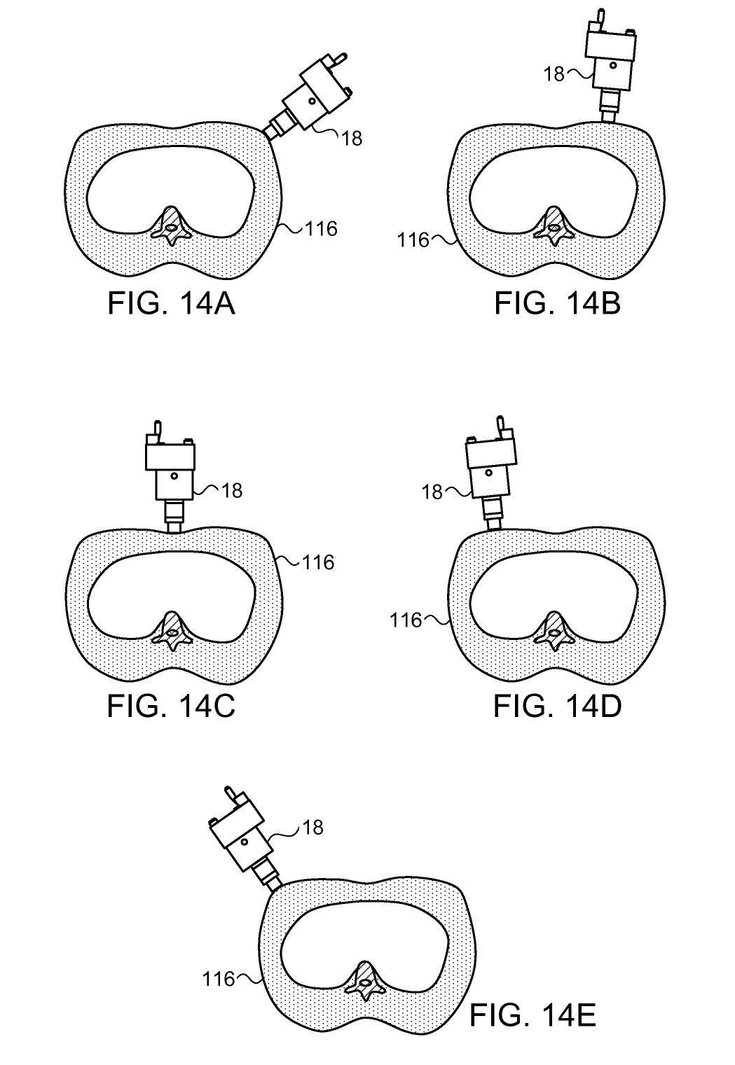

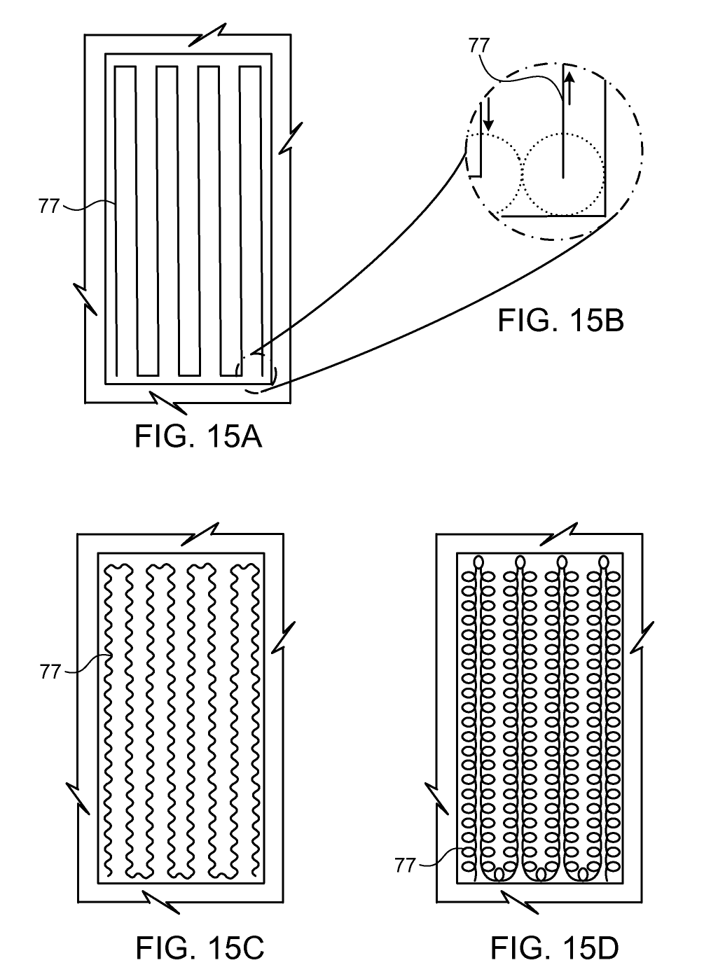

FIGS. 14A-14E illustrate the subsequent motion of holder 18 and the applicator mounted therein during a first pass of the treatment process. As seen here, the processes of self-correction of both orientation and contact pressure relative to the skin continue throughout the motion, thereby maintaining proper alignment of the applicator and a safe and effective level of contact pressure throughout the motion. Subsequent passes of the treatment will then continue along adjacent strips of the skin, as defined in the treatment path parameters, for example as described above.

It will be appreciated that the above descriptions are intended only to serve as examples, and that many other embodiments are possible within the scope of the present invention as defined in the appended claims.

What is claimed is:

1. A robotic system for delivering energy for medical or cosmetic treatment of a region of skin of a subject, the system comprising:
(a) a multi-axis robotic arm mounted on a base and terminating at a holder;
(b) an applicator for delivering energy to the skin, said applicator being mechanically associated with said holder;
(c) at least three range sensors associated with said holder and deployed to generate outputs indicative of a distance to a corresponding at least three spaced apart locations on the surface of the skin; and
(d) a controller associated with said robotic arm and said range sensors, said controller including at least one processor, said controller being configured to:
(i) process said outputs from said at least three range sensors to determine an orientation of said holder relative to the skin, and
(ii) actuate said robotic arm to adjust an orientation of said holder such that an orientation of said applicator relative to the skin is maintained within a predefined range of desired angles.

2. The system of claim 1, wherein said controller is configured to actuate said robotic arm so as to move said holder along a substantially orbital path about a point of contact between said applicator and the skin.

3. The system of claim 1, wherein said predefined range of desired angles lies within 20 degrees of perpendicular.

4. The system of claim 1, wherein said range sensors are non-contact range sensors.

5. The system of claim 1, wherein said applicator is retractably mounted relative to said holder so as to retract under contact pressure with the skin, and wherein one of said range sensors is implemented as a position sensor deployed for sensing a position of said applicator relative to said holder.

6. The system of claim 1, wherein said applicator is retractably mounted relative to said holder, and wherein said head further comprises a resilient biasing arrangement deployed so as to bias said applicator against the skin.

7. The system of claim 6, wherein said resilient biasing arrangement includes a counterweight deployed to substantially cancel variations in said bias due to the weight of said applicator.

8. The system of claim 1, wherein said applicator is referred to as a first applicator, and further comprising a second applicator, wherein said first and second applicators are configured to be interchangeably mounted relative to said holder, and wherein said first and second applicators are each configured to provide a distinct treatment modality selected from the group consisting of: laser light; non-laser light; RF irradiation; microwave irradiation; ultrasound treatment; and mechanical manipulation.

9. The system of claim 1, wherein said applicator is part of an RF irradiation treatment system.

10. The system of claim 1, wherein said base of said robotic arm is mounted slidably on a substantially linear track.

11. The system of claim 1, further comprising at least one user-operable control for controlling a position of said holder, and wherein said controller is configured to:
(a) allow a user to maneuver said holder so as to bring said holder into alignment with a location on the skin; and
(b) receive from the user a point designation input designating the location on the skin as a boundary point at least partially defining a treatment region.

12. The system of claim 11, wherein alignment of said holder with the location on the skin is defined by alignment of a visible light beam projected from said holder with the point on the skin without requiring contact with the skin, and wherein said controller is further configured, after receipt of said point designation input, to actuate said robotic arm to move said applicator into contact with the skin at the boundary point.

13. A method for delivering energy for medical or cosmetic treatment of a region of skin of a subject, the system comprising:
(a) providing a multi-axis robotic arm mounted on a base and terminating at a holder;
(b) providing an applicator for delivering energy to the skin, said applicator being mechanically associated with said holder;
(c) providing at least three range sensors associated with said holder and deployed to generate outputs indicative of a distance to a corresponding at least three spaced apart locations on the surface of the skin;
(d) processing said outputs from said at least three range sensors to determine an orientation of said holder relative to the skin, and
(e) actuating said robotic arm to adjust an orientation of said holder such that an orientation of said applicator relative to the skin is maintained within a predefined range of desired angles.

14. The method of claim 13, wherein said adjusting an orientation is performed so as to move said holder along a substantially orbital path about a point of contact between said applicator and the skin.

15. The method of claim 13, wherein said predefined range of desired angles lies within 20 degrees of perpendicular.

16. The method of claim 13, wherein said range sensors are non-contact range sensors.

17. The method of claim 13, wherein said applicator is retractably mounted relative to said holder so as to retract under contact pressure with the skin, and wherein one of said range sensors is implemented as a position sensor deployed for sensing a position of said applicator relative to said holder.

18. The method of claim 13, wherein said applicator is retractably mounted relative to said holder, and wherein said head further comprises a resilient biasing arrangement deployed so as to bias said applicator against the skin.

19. The method of claim 18, wherein said resilient biasing arrangement includes a counterweight deployed to substantially cancel variations in said bias due to the weight of said applicator.

20. The method of claim 13, wherein said applicator is referred to as a first applicator, and further comprising a second applicator, wherein said first and second applicators are configured to be interchangeably mounted relative to said holder, and wherein said first and second applicators are each configured to provide a distinct treatment modality selected from the group consisting of: laser light; non-laser light; RF irradiation; microwave irradiation; ultrasound treatment; and mechanical manipulation.

21. The method of claim 13, wherein said applicator is part of an RF irradiation treatment system.

22. The method of claim 13, wherein said base of said robotic arm is mounted slidably on a substantially linear track.

23. The method of claim 13, further comprising:
(a) allowing a user to maneuver said holder so as to bring said holder into alignment with a location on the skin; and
(b) receiving from the user a point designation input designating the location on the skin as a boundary point at least partially defining a treatment region.

24. The method of claim 23, wherein alignment of said holder with the location on the skin is defined by alignment of a visible light beam projected from said holder with the point on the skin without requiring contact with the skin, and wherein said controller is further configured, after receipt of said point designation input, to actuate said robotic arm to move said applicator into contact with the skin at the boundary point.

25. A method for designating a treatment region on the skin of a subject for therapeutic or aesthetic treatment, the method comprising the steps of:
(a) allowing manual manipulation of a treatment head of a robotic skin treatment system so as to bring the treatment head into alignment with a location on the skin; and
(b) receiving a point designation input designating the location on the skin as a boundary point at least partially defining a treatment region.

26. The method of claim 25, wherein said alignment is defined by alignment of a visible light beam projected from said treatment head with the point on the skin without requiring contact with the skin, wherein said method further comprises, after receipt of said point designation input, moving said treatment head into contact with the skin at the boundary point.

27. The method of claim 25, wherein said steps of allowing manual manipulation and receiving a point designation input are repeated so as to designate a plurality of boundary points, the method further comprising:
(a) defining a boundary line derived from said plurality of boundary points; and
(b) defining a scanning pattern bounded by said boundary line, thereby defining a treatment region on the skin.
